(19) United States
(12) Patent Application Publication
(10) Pub. No.: US 2002/0032381 A1
(43) Pub. Date: Mar. 14, 2002

(54) Method and apparatus for ultrasonic tissue investigation

(76) Inventors: Mary Dyson, Berkhamsted (GB); Bonita Weyrauch, Drexil Hill, PA (US); Paul R. Quintavalls JR., Haddonfield, NJ (US)

Correspondence Address: KNOBLE & YOSHIDA, EIGHT PENN CENTER, SUITE 1350, 1628 JOHN F KENNEDY BLVD, PHILADELPHIA, PA, 19103, US

(21) Appl. No.: 09/990,014

(22) Filed: Nov. 21, 2001

Related U.S. Application Data
(63) Continuation of application No. 09/510,263, filed on Feb. 22, 2000.
Application No. 09/510,263 is a continuation-in-part of application No. 08/894,791, filed on Feb. 9, 1998, now Pat. No. 6,073,045.

(51) Int. Cl. A61B 8/00

(52) U.S. Cl. 600/443; 600/439

(57) ABSTRACT

A method of evaluating and treating a burn patient includes steps of scanning an area of damaged skin with an ultrasonic scanner that has a resolution that is capable of producing an image that is of sufficient quality to determine whether the area of damaged skin has suffered a partial thickness burn or full thickness burn, using the image to determine whether the area of damaged skin has suffered a partial thickness burn or full thickness burn, and then effecting a skin graft on the area of damaged skin if a determination is reached that the burn is a full thickness burn.

This is a continuation of Ser. No. 09/510,263, filed Feb. 22, 2000, which in turn is a continuation-in-part of Application Ser. No. 08/894,791, filed Feb. 9, 1998, the entire disclosures of which are hereby incorporated as if set forth fully herein.

### BACKGROUND OF THE INVENTION

1. Field of the Invention

The present invention relates to an apparatus for an investigation of tissue based on the emission and reception of ultrasound.

2. Description of the Related Technology

It is known to use ultrasound to carry out investigations of the human body and other animal bodies. In these cases, an ultrasonic transducer is linked acoustically to the skin of a patient or other subject, optionally with the introduction of an appropriate coupling and lubricating medium. Ultrasonic pulses transmitted into the patient are then reflected from reflecting surfaces at acoustic interfaces between and/or within the various layers of tissue within the patient. The reflected pulses are received by the transducer and signals representative of the pulses are generated and combined by appropriate computing means to enable a visual representation of the zone of treatment of the patient to be recreated. One example of the use of such techniques is ultrasonic scanning of a fetus in a pregnant mother's womb.

Human and other animal tissues are arranged in layers from superficial to deep usually comprising the outermost layer of epidermis which is subdivided into acoustically different confified and noncornified strata, followed by papillary and reticular layers of dermis, beneath which lies a layer of fat and then other tissues such as tendon, ligament, muscle and bone. At each of the interfaces between and/or within these various layers, a proportion of the ultrasonic input will be reflected and can be received to generate a visually identifiable picture of the condition at any one particular interface. This noninvasive technique enables and aids diagnosis of any acoustically distinguishable disorder of the skin or underlying tissue.

However, presently available techniques cannot always give a clear enough view of any likely problem, and it is therefore an object of the present invention to provide an apparatus which will give an improved representation of the condition of a patient or other subject at a desired location within or beneath the skin.

### SUMMARY OF THE INVENTION

According to a first aspect of the present invention there is provided an apparatus for ultrasonic tissue investigation comprising ultrasonic transducer means adapted to emit pulsed emissions into tissue, means so to move said transducer means as to scan an area of tissue to be investigated, means to receive signals reflected from interfaces between and/or within tissue layers, means to convert said received signals into a visual image of the tissue, and means to display said visual image, wherein said emissions of ultrasonic radiation are so pulsed that each pulse has a very rapid fall back period.

Preferably there are provided means to analyze the data from which these images are produced.

Preferably, the received signals are split into positive and negative part signals, each of which is separately amplified by log compressor means, with the amplified signals being recombined to give an input to said means to convert said recombined signals into a visual image.

The means to move said ultrasonic transducer may be a stepper motor adapted to move said ultrasonic transducer within an area having a travel of up to approximately 15 mm, using a transducer of diameter up to approximately 6 mm.

Each scan of the area may involve a plurality of pulses, having a pulse repetition frequency in the region of 1 ms, each pulse being of duration less than 50 ns.

According to a second aspect of the present invention there is provided a method of tissue investigation comprising scanning an area of tissue using an apparatus as described above.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 is a cross-sectional view of a probe that is constructed according to a preferred embodiment of the invention;

FIG. 2 is a diagram of the circuit for the probe pulser and preamplifier of the present Invention;

FIG. 3 is a circuit diagram for the log amplifier system of the present invention;

FIG. 4 shows the system in a schematic form;

FIG. 5 shows schematically the electronic systems of the probe; and

FIG. 6 shows schematically the electronic systems of the main RF amplifier.

### DETAILED DESCRIPTION OF THE PREFERRED EMBODIMENT(S)

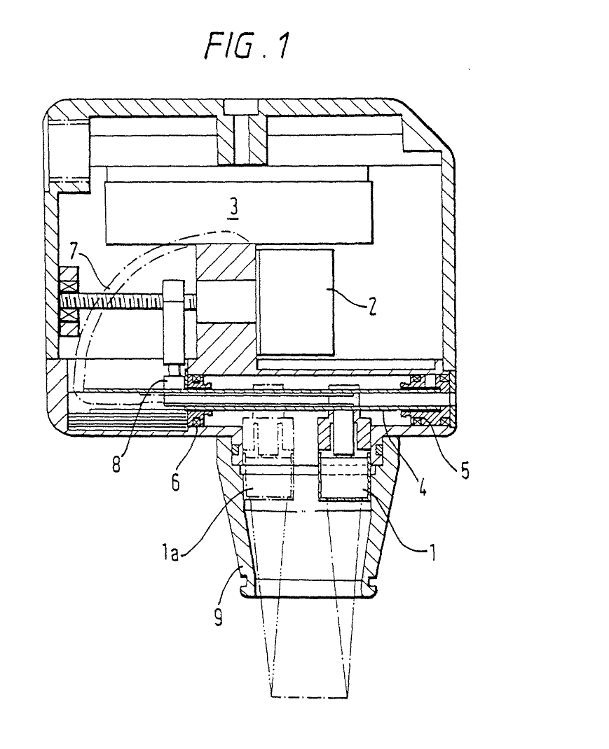

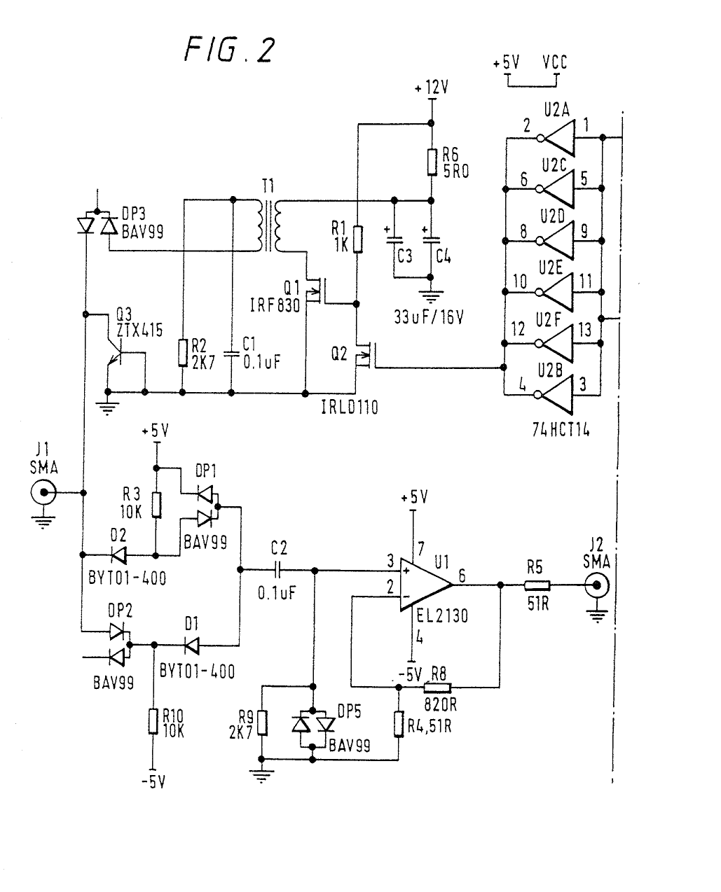

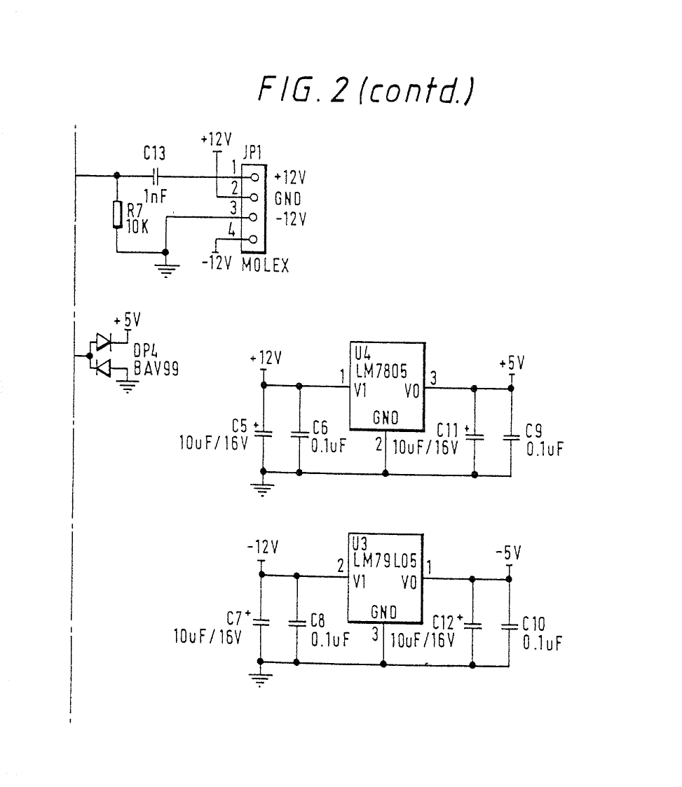

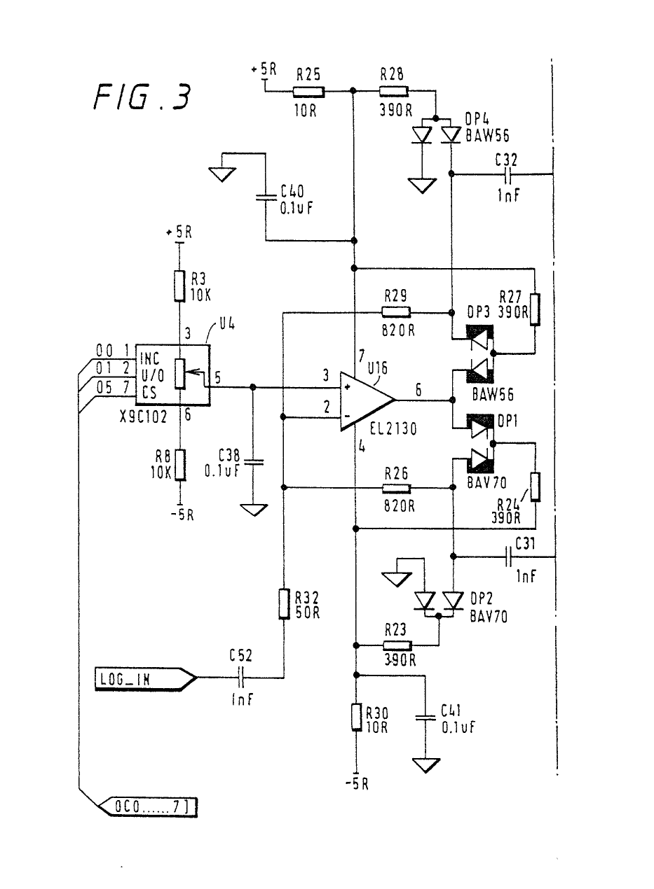

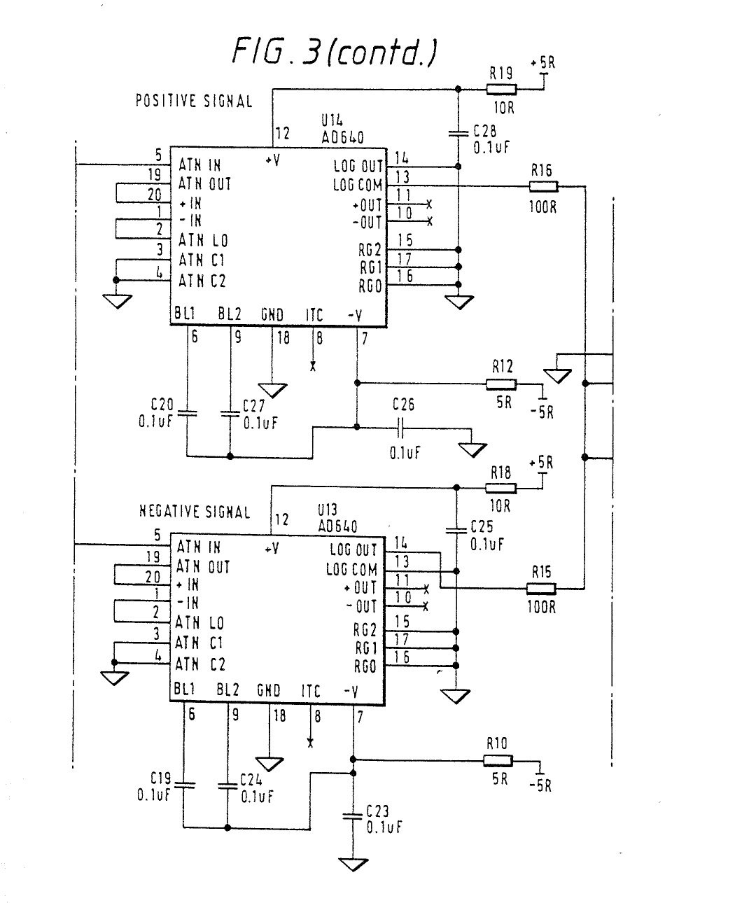

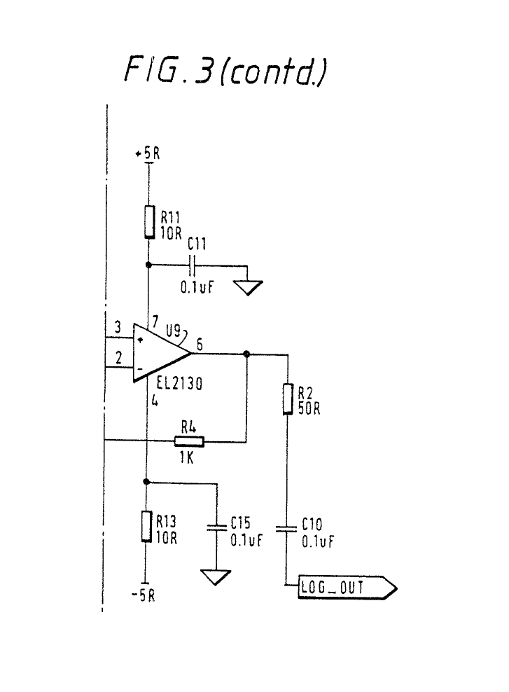

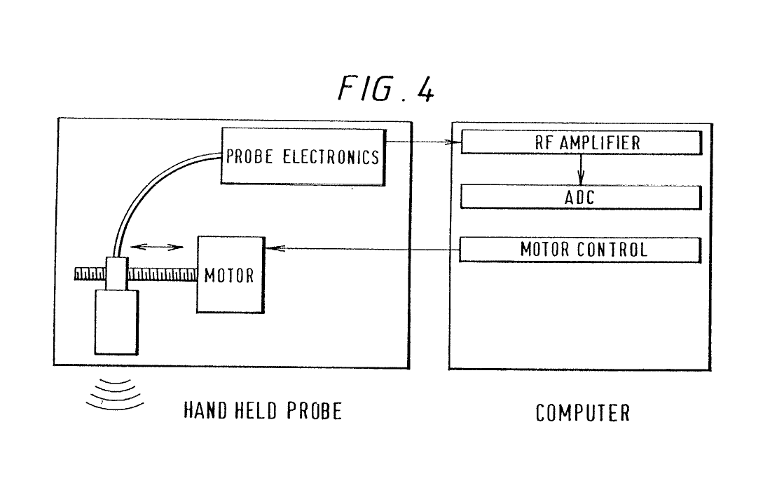

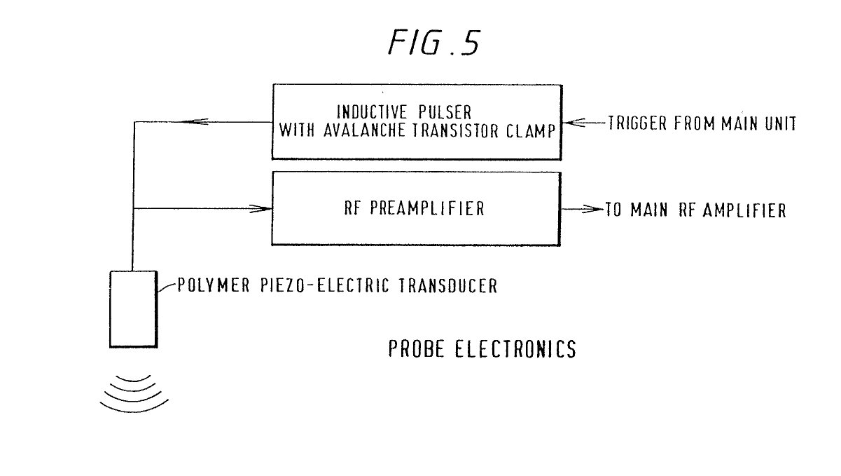

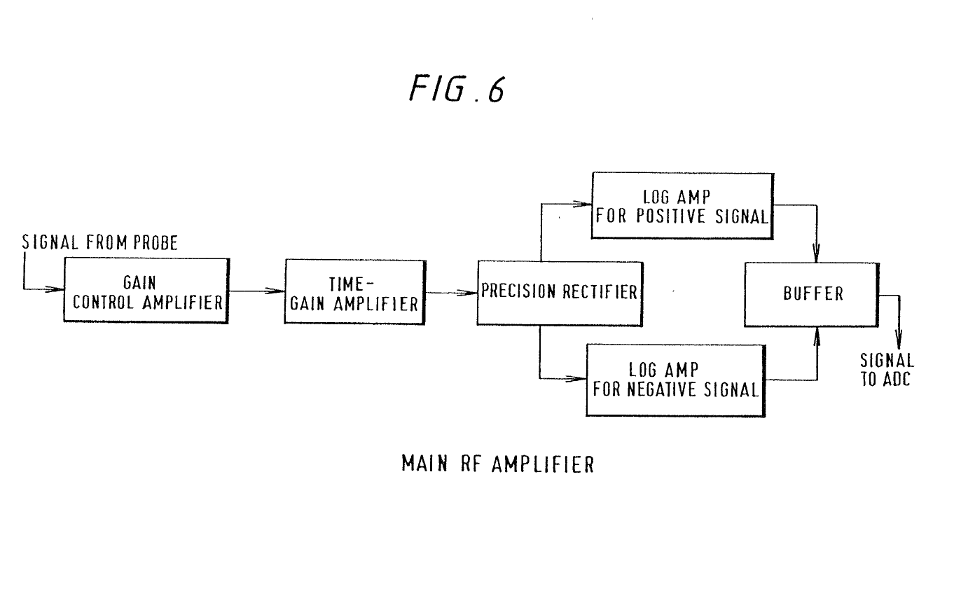

Referring now to the drawings, wherein like reference numerals designate corresponding structure throughout the views, and referring in particular to FIG. 1, an apparatus according to the preferred embodiment of the invention is constructed and arranged for application to an area of skin of a patient, another subject or animal hide. The apparatus comprises a transducer 1, which may be moved by means of a stepper motor 2 to scan an area of tissue on or under that particular area of skin. The motor 2 drives shaft 4, which is supported between an external shift bearing 5 and an internal shaft bearing 6, and the transducer 1 is movable between positions 1 and 1a as shown in FIG. 1. The transducer is driven along the shaft by means of flexible drive 7, although connection may also be made through link arm 8.

The transducer 1, housed within cone 9, has preferably a piezoelectric polymer element capable of emitting a single cycle pulse at a frequency of between 10 and 50 MHz. The preferred center frequency is in the region of 20 MHz and, as will be described below, the probe transducer is activated at a high voltage, and the system is adapted to cause an ultra-fast rise and fall time pulse. The sharp signals given thereby enable a better reception of the reflected signals.

The control electronics of the system are housed in area 3 and are described in more detail below. Of these, the probe electronics contain a pulse generator for energizing the ultrasound transducer and a preamplifier for the returning signal. The time gain compensator and motor control unit contains the main signal amplifier and the control and drive electronics for the probe motor. This may be connected to a compatible computer along with an analogue to digital converter board. In the probe electronics there are two parts. First the pulser, which generates an ultra-fast signal of rise and fall time>30 kV/μs, very short duration (<30 ns) and high voltage (>300V). The pulse is produced from a single low voltage (+12 volt) supply to the board. The pulse is created by the back emf of a small inductor acting as an impedance transformer with an avalanche transistor to limit the pulse duration and to produce an ultra fast fall time for the pulse. Power is supplied to the circuit through pins 2 and 3 of JPI. Pulse triggering is generated by a 3 μs 5-volt positive supplied to pin 1 of JPI. This causes the inputs to U2 of the 74HCT14 hex inverting Schmitt triggered buffer to go high for 3 μs. The turn-on speed of Q1 is limited by R1 and the gate capacitance of Q1. This prevents a significant pulse from the in-rush current into T1, which saturates within 3 μs. On the falling edge of the trigger pulse the outputs of U2 go high turning Q3 on quickly which turns off Q1 in less than 10 ns. This causes a back emf pulse of in excess of 600 volts to be generated across T1. This is stepped down to a 300-volt pulse at the output of T1. The diodes in DP3 conduct and the pulse is fed to the transducer connected to J1. When the output pulse voltage reaches greater than 300 volts Q3 breaks down in an avalanche-shorting transformer and limits the pulse duration.

In this respect there is provided a preamplifier with input protection, which consists of a standard current mode opamp with the input protected by a diode bridge. This amplifier has a voltage gain of 17 (13.3 dB).

The received signal, having been preamplified, is fed to a time gain compensation circuit to allow for attenuation through the various layers of tissue being investigated. An amplifier consists of five stages. The first stage is a variable gain amplifier with a −10 dB to +30 dB control. This acts as overall gain control for the system. The second stage is a variable gain amplifier and ramp generator with a 0 to +30 dB control range that is ramped at a controlled rate and acts as the time gain compensation. The third stage is a precision rectifier that splits the signal into positive and negative parts of the signal, and for the fourth stage, a log compressor consisting of two 50 dB log amps. The output of these two log amps is recombined in the fifth stage and buffered to give a 50 ohm output for input to the analogue/digital converter.

Secondly, a motor control comprises a bipolar stepper motor drive using MOSFET transistors with phase sequencing provided by a specially programmed PAL. The step rate and step count is controlled by the host computer through an industrial standard S354 counter/timer IC.

By keeping apart the positive and negative parts of the return signal, and then combining them after amplification, it has been found that the signal has an improved range and discrimination when transferred to an analogue digital converter and thence to the computing means and visual display means.

Value can be added to the visual display by subjecting the returned signals either to a process of fractal analysis, wavelet analysis or a fast Fourier transform. Fractal analysis comprises representing the region of interest of the skin and underlying tissue as a three dimensional landscape, with lateral and axial dimensions on a horizontal plane and the intensity of the image (0-256) on a vertical axis. The area of landscape can then be mapped using flat disc shaped structuring elements with no height in a grey scale dimension and using techniques of mathematical morphology, the surface area of the image can be measured at different resolutions by removing features of less than a particular size.

According to the method, this can be performed for resolutions between 1 pixel up to 20 pixels. At any given resolution, the rate of change of the surface area with respect to resolution is related to the estimated fractal dimension at that resolution. The set of estimated fractal dimensions up to resolutions of 20 pixels defines the fractal signature.

Experiments show that, in using this system, various areas of tissue have a distinctive signature. For example, signatures from forehead tissue and regions of the hand lie particularly close together throughout pixel size, especially in the range of 2-5 pixels, peaking with fractal dimensions between 3.5 and 4.0 at 5 pixels. Other parts of the body gave different signatures, for example a scan of heel tissue peaks at 9 pixels with a fractal dimension of 3.2.

Similar experiments using fast Fourier transforms (FFTs) have shown that the heel sample shows the lowest overall curve amplitude, whilst the samples from equivalent tissues of the hand show a remarkable similarity in curve shape and size, regardless of the part of the hand from which the samples were taken. Samples taken from forehead tissue show greatest amplitude at the first peak, with the second peak lower and showing the prevalence of narrow bands.

Samples from hand and heel tissues show second peaks larger than the first ones, and the prevalence of wide bands.

It is thought that fractal analysis will give different signatures for normal and damaged tissues, and the information can be stored for use as a comparison in all future studies. A databank built up in this way would be able to give more immediate attention to any abnormalities in the tissue being examined.

Apparatus embodying the invention will find use in identifying and diagnosing tumors, injuries and any other abnormal condition up to depth of 3-5 cm below the skin surface being investigated. Such noninvasive investigation is obviously a benefit to the patient and the apparatus provides a means of carrying out such investigation quickly, and with the advantage of giving clear images of any problem which may be encountered within the tissue surveyed.

One further use of the invention is in the testing of hides, sheepskins, and other materials used for commercial purposes such as clothing, footwear or upholstery. In this case, the value of the hide will depend upon its dermal perfection. Dermal imperfections are frequently not revealed until the completion of dyeing, and may be are further masked by the presence of hair or wool. The apparatus of the present invention can be used to scan a hide from an “inside” (deep) surface thereof and determine whether imperfections are likely to appear on the opposite “outside” surface following dyeing once the hair or wool has been removed.

The interoperative use of 20 Mhz ultrasound B-scans to image burns is another important aspect of the invention that will now be described. It is known that the early excision and grafting of full thickness or third degree burns decreases morbidity and mortality. In contrast, partial thickness or second degree burn wounds can generally reepithelialize without excision and grafting. If errors are made in assessing the depth of burns this can lead to prolonged hospitalization, and to unnecessary and damaging excision of the beds of partial thickness wounds, converting them to full thickness wounds requiring grafting.

In 1986 Brink et al reported the successful use of high-frequency (18.5 MHz) ultrasonic imaging to noninvasively quantitate burn depth in pig skin. In contrast Wachtel et al, also in 1986, concluded that high-frequency ultrasound as practiced then was “of no practical value to the burn surgeon for differentiating precisely between the depth of a deep dermal wound and a full skin thickness thermal injury”. By 1989 Bauer and Sauer reported the sonographical demonstration with 10 MHz ultrasound of alterations in the epidermis and dermis in deep dermal scald wounds displayed by “different echo reflections”. Cammarota et al, in a review published in 1998, mentions that high frequency ultrasound can be used “in the follow-up of focal burns”. However, the scanners used in these studies apparently lacked the resolution and clarity that would be necessary to identify the precise depth to which burn damage of varying degrees extends. Accordingly, the studies identified little that would be of use to a clinician such as a dermatologist or surgeon treating a burn victim.

One important aspect of the invention is the interoperative use of high frequency (20 MHz or greater) ultrasound imaging that is performed by the scanner according to the invention as described above to assist the surgeon in deciding which parts, if any, of a burn wound require excision ancillary to grafting. Aspects of the scanner that make it suitable for use in surgery include the portability of the unit, the ability of the scanner to be sheathed in sterile material, the resolution that the scanner provides, which can be as low as 35 microns, its ease of use and the speed of data acquisition. Another advantage is that the scanner has the ability to scan through hydrocolloid and semi-occlusive film dressings.

Areas of uniqueness that the present invention offers to conventional burn evaluation and management include the ability to scan the first millimeter of skin throughout an extended evaluation area at a high resolution that is clinically adequate to ascertain whether the skin has suffered at any one location a partial or a full thickness burn. This gives a clinician the ability to discriminate viable tissue from nonviable tissue by evaluating such visible effects as separation of epidermis from dermis, which is a precursor to blistering; to view the extent of coagulation of protein within papillary and reticular layers of the dermis; to view whether damage extends into the hypodermis; and to view and evaluate fluid collection within the layers of the dermis and hypodermis resulting from microvascular permeability changes associated with thermal injury. The ability to evaluate a display that reveals one or more of these effects permits differentiation of first, second (“partial thickness”) and third (“full thickness”) degree burns. This information can be used to help decide whether a skin graft is indicated for a particular patient, and the area or areas on the patient for which such grafts would be necessary.

Another application for the use of the scanner described above is in the assessment of skin grafts and flaps. The most common reason for failure of a skin graft is fluid accumulation between the graft and the graft bed. This can be either serum or active bleeding. The scanner provides the ability to determine whether there is any fluid at the interface between the graft and the bed. Early detection of this fluid is critical for the survival of the graft or flap. The scanner also allows for imaging of the wound bed within 1 cm. of the surface. This information may allow for monitoring the extent of revascularization in this tissue, abscess formation, edema or other indicators of potential graft failure. The scanner would also be of value in assessing revascularization with porcine grafts as well as synthetic skin. This information may allow for better a determination if and when skin grafting would be necessary.

Most surgeons prefer that the initial surface dressing remain in place for at least 5 to 7 days. The scanner allows for imaging through some dressings including hydocolloids, calcium alginates and films. This would allow the clinician to image the graft, bed and graft-bed interface without disturbing the surface dressing.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method of evaluating and treating a burn patient, comprising steps of:
(a) scanning an area of damaged skin with an ultrasonic scanner that has a resolution that is capable of producing an image that is of sufficient quality to determine whether the area of damaged skin has suffered a partial thickness burn or full thickness burn;
(b) using the image to determine whether the area of damaged skin has suffered a partial thickness burn or full thickness burn; and
(c) effecting a skin graft on the area of damaged skin if a determination is reached in step (b) that the burn is a full thickness burn.

2. A method according to claim 1, wherein step (b) is performed by using the image to determine if burn-induced separation of epidermis from dermis has occurred in the area of damaged skin.

3. A method according to claim 1, wherein step (b) is performed by using the image to determine the extent of coagulation of protein that has occurred within papillary and reticular layers of the dermis in the area of damaged skin.

4. A method according to claim 1, wherein step (b) is performed by using the image to view and evaluate fluid collection within the layers off the dermis and hypodermis resulting from microvascular permeability changes caused by thermal injury.

5. A method according to claim 1, further comprising a step (d) of using the scanner to evaluate the quality of a skin graft that is performed in step (c).

6. A method according to claim 5, wherein step (d) comprises using the scanner to detect any fluid that has accumulated at the interface between the graft and the graft bed.

7. A method according to claim 5, wherein step (d) comprises using the scanner to view the degree of revascularization that has occurred after the graft has been effected.

8. A method according to claim 5, wherein step (d) comprises using the scanner to scan through a dressing without removing the dressing.

9. A method according to claim 9, wherein said dressing is a hydrocolloid dressing.

10. A method according to claim 9, wherein said dressing is a calcium alginate dressing.

11. A method according to claim 9, wherein said dressing is a film dressing.

12. A method of treating an area of skin that has suffered a full-thickness burn, comprising steps of:
(a) determining whether an area of skin has suffered a full-thickness burn;
(b) effecting a skin graft on the area of skin if a determination is reached in step (a) is that the burn is a full thickness burn; and
(c) using an ultrasonic scanner to evaluate the quality of a skin graft that is performed in step (b).

13. A method according to claim 12, wherein step (c) comprises using the scanner to detect any fluid that has accumulated at an interface between the graft and the graft bed.

14. A method according to claim 12, wherein step (c) comprises using the scanner to view the degree of revascularization that has occurred after the graft has been effected.

15. A method according to claim 12, wherein step (c) comprises using the scanner to scan through a dressing without removing the dressing.

16. A method according to claim 15, wherein said dressing is a hydrocolloid dressing.

17. A method according to claim 15, wherein said dressing is a calcium alginate dressing.

18. A method according to claim 15, wherein said dressing is a film dressing.
